(19) United States
(12) Patent Application Publication
(10) Pub. No.: US 2010/0078032 A1
(43) Pub. Date: Apr. 1, 2010

(54) CONDOM PACKAGING

(76) Inventor: Corey John Sarros, San Luis Obispo, CA (US)

Correspondence Address: DAVIS WRIGHT TREMAINE LLP/Los Angeles, 865 FIGUEROA STREET, SUITE 2400, LOS ANGELES, CA, 90017-2566, US

(21) Appl. No.: 12/571,028

(22) Filed: Sep. 30, 2009

Related U.S. Application Data
(60) Provisional application No. 61/101,289, filed on Sep. 30, 2008.

(51) Int. Cl. A61F 6/04; B65D 85/08

(52) U.S. Cl. 128/844; 206/69

(57) ABSTRACT

The present subject matter relates to a condom packaging apparatus capable of reducing the exposure of physical damage to the condom, and eliminating or significantly reducing the misapplication and misfitment of a condom onto the end user's penis. Furthermore, the present subject matter relates to methods for applying a condom to a penis capable of reducing the exposure of physical damage to the condom, and eliminating or significantly reducing the misapplication and misfitment of a condom onto the end user's penis.

### CROSS REFERENCE TO RELATED APPLICATIONS

This application claims the benefit of priority from U.S. Provisional Application Ser. No. 61/101,289, filed Sep. 30, 2008.

### FIELD OF PRESENT SUBJECT MATTER

This present subject matter relates to packaging for condoms. More specifically the present subject matter relates to a single condom packaging designed to allow easy application of the condom upon an erect penis, without incidents of mishandling and/or misuse of the condom.

### BACKGROUND

All publications herein are incorporated by reference to the same extent as if each individual publication or patent application was specifically and individually indicated to be incorporated by reference. The following description includes information that may be useful in understanding the present subject matter. It is not an admission that any of the information provided herein is prior art or relevant to the presently claimed present subject matter, or that any publication specifically or implicitly referenced is prior art.

Condoms have been used for over 380 years. A condom is a barrier device most commonly used during sexual intercourse to reduce the likelihood of pregnancy and spreading sexually transmitted diseases, such as gonorrhea, syphilis, and HIV. A condom is effective when placed on the erect penis prior to intercourse, and physically blocks ejaculated semen from entering the body of a sexual partner. Because condoms are waterproof, elastic, and durable, they are also used in a variety of secondary applications, which includes collection of semen for use in infertility treatment.

The effectiveness of condoms, as with most forms of contraception, can be assessed in two ways. Perfect use effectiveness rates only calculate the use of a condom by individuals who use the condom properly and consistently. Actual use effectiveness rates take into account the use of condoms by a wide range of individuals who may use the condom incorrectly, thus reducing it's effectiveness as a contraceptive. The typical use pregnancy rate among condom users varies depending on the population being studied, ranging from 10-18% per year. The perfect use pregnancy rate of condoms is 2% per year. As the variance between perfect use and actual use is quite large, a greater degree of emphasis has been placed in educating people in the proper method for using a condom, as this would lead to less incidents of failure.

In typical use cases, condom failure is attributed mostly to breaks or tears in the condom due to physical damage. Damage to the condom caused when opening the package is attributable for 0.4% to 2.3% of condom failures, while failure due to slippage in the condom (due to misuse) is between 0.6% and 1.3% (National Institute of Allergy and Infectious Diseases; National Institutes of Health, Department of Health and Human Services (Jul. 20, 2001). “Workshop Summary: Scientific Evidence on Condom Effectiveness for Sexually Transmitted Disease (STD) Prevention).

Existing designs for packing of condoms require the end user to tear apart the packaging with their hands or, if necessary, teeth, thus exposing the condom to possible damage. In addition, existing condom package designs require additional handling of the condom, prior to application on the penis, increasing the possibility of damage by end users nail's, hands and such.

Misapplication of the condom is yet another cause for tears and punctures in a condom leading to failure. Most condoms are supplied with lubrication applied to the outer layer of the condom. The lubricated outer layer helps decrease friction during vaginal or anal sex, which can otherwise rupture the condom. In application an end users may fit the condom to the penis inside-out, leading to condom failure due to stretching and tearing of the condom as lubricants designed to be on the outside of the condom are now in contact with the penis. Additionally, the inside-out placement of the condom causes the lubricants, intended to be on the outside of the condom, to dislodge the condom from the end users penis, increasing semen exposure.

Accordingly, there exists a need in the art for a condom packaging apparatus capable of reducing the exposure of physical damage to the condom. In addition, there exists a need in the art for a condom packaging apparatus capable of eliminating misapplication and misfitment of the condom onto the end user's penis. Furthermore, there exists a need in the art for a method of applying a condom to a penis capable of reducing the exposure of physical damage to the condom. Finally, there exists a need in the art for a method of applying a condom to a penis capable of eliminating misapplication and misfitment of the condom onto the end user's penis.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a top perspective view of the condom packaging in accordance with an embodiment of the present subject matter.

FIG. 2 depicts an exploded top perspective view of the condom packaging in accordance with an embodiment of the present subject matter.

FIG. 3 depicts a top diaphanous view of the condom packaging in accordance with an embodiment of the present subject matter.

FIG. 4 depicts a side sectional view of the condom packaging in accordance with an embodiment of the present subject matter.

FIG. 5 depicts a side sectional view of the condom packaging in accordance with an embodiment of the present subject matter.

### DETAILED DESCRIPTION OF THE SUBJECT MATTER

All references cited herein are incorporated by reference in their entirety as though fully set forth. Unless defined otherwise, technical and scientific terms used herein have the same meaning as commonly understood by one of ordinary skill in the art to which this present subject matter belongs. One skilled in the art will recognize many methods and materials similar or equivalent to those described herein, which could be used in the practice of the present subject matter. Indeed, the present subject matter is in no way limited to the methods and materials described.

“Plastic lid” as used herein refers to a means for opening the condom packaging at the removable top. The lid serves as the cover or seal, usually one that completely closes the object. As referenced herein the lid is constructed of a plastic material.

“Pop-top” as used herein refers to a means for opening the condom packaging at the removable top. The pop-top involves a integral rivet and pull-tab version (also known as rimple or ring pull), which had a ring attached at the rivet for pulling, and which would come off completely to be discarded. The pop-top was invented in 1962, by Ermal Cleon Fraze, and received U.S. Pat. No. 3,349,949.

“Pull-Tap” as used herein refers to a means for opening the condom packaging at the removable top. The pull-tab involves a scored lid with a pull-tab. The pull-tab can be leveraged to push the scored region into the can, opening up a hole.

The present subject matter relates to methods and apparatus for housing and applying a condom to a penis. More specifically, the present subject matter discloses a condom packaging apparatus that is capable of reducing the exposure of physical damage to the condom, eliminating or significantly reducing the misapplication and misfitment of a condom onto the end user's penis, and increasing the functional use of a condom. Furthermore, the present subject matter discloses methods for applying a condom to a penis capable of reducing the exposure of physical damage to the condom, and eliminating or significantly reducing the misapplication and misfitment of a condom onto the end user's penis.

The subject matter apparatus comprises a condom package for dispensing a condom, wherein the condom package comprises a cylindrical container with a round bottom and a cylindrical side wall, wherein the round bottom is flat and has a diameter configured to encompass the diameter of a condom, and the cylindrical side wall has a height configured to encompass the height of a rolled condom, such that the container is capable of housing a rolled condom. The apparatus further comprises a removable top for sealably enclosing the condom in the container. The removable top is attached at the upper rim of the cylindrical side wall of the container, and the removable top may have an edge extending beyond the rim of the side wall for ease of removing the removable top.

In various embodiments, the removable top and container may be independently constructed of a material selected from a plastic, an elastomer, rubber, an elastic polymer, a metal alloy, derivatives thereof and combinations thereof.

In further embodiments, the operational element of the removable top of the condom package may be a pop-top, a pull-tab, a plastic lid, a peel-off top and combinations thereof. Furthermore, the removable top may be luminous, for ease of operation in the dark. In yet another embodiment, the exterior surface of the condom may contain a lubricant for easier and more compliant penetration during sex.

The present subject matter also discloses methods for applying a condom to a penis capable of reducing the exposure of physical damage to the condom, and eliminating or significantly reducing the misapplication and misfitment of a condom onto the end user's penis. In one embodiment, the method comprises providing a condom package for dispensing a condom, wherein the condom package comprises a cylindrical container with a round bottom and a cylindrical side wall, wherein the round bottom is flat and has a diameter configured to encompass the diameter of a condom, and the cylindrical side wall has a height configured to encompass the height of a rolled condom, such that the container is capable of housing a rolled condom, with the condom package further comprising a removable top, sealably attached at the upper rim of the cylindrical side wall of the container, for enclosing the condom in the container. The method further includes removing the removable top of the condom package, exposing the rolled condom in the container, and placing the rolled condom and container on the penis with the exposed condom resting atop the head of the penis. The rolled condom is unrolled towards the base of the penis, thereby covering the penis with the condom, and the container is discarded.

The rolled condom is situated in the container such that the condom may only be unrolled towards the base of the penis, and such that the condom may only cover the penis in the proper configuration and not inside-out.

In various embodiments, the removable top and container may be independently constructed of a material selected from a plastic, an elastomer, rubber, an elastic polymer, a metal alloy, derivatives thereof and combinations thereof.

In further embodiments, the operational element of the removable top of the condom package may be a pop-top, a pull-tab, a plastic lid and combinations thereof. The removable top may be sealably attached to the container by an adhesive or other means known in the art. Furthermore, the removable top may be luminous, for ease of operation in the dark. In yet another embodiment, the exterior surface of the condom may contain a lubricant for easier and more compliant penetration during sex.

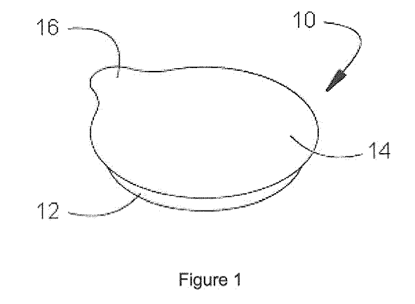

FIG. 1 illustrates a top perspective view of an embodiment of the present subject matter condom packaging 10, comprising a container 12 and a removable top 14 attached to the container 12. FIG. 1 further depicts an edge 16 protruding beyond the end of the container 12 for ease of handling and removal of the removable top 14 by an end user.

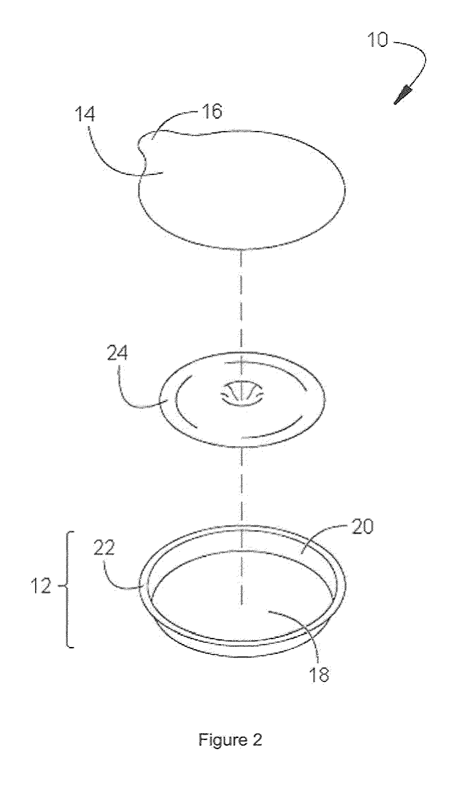

FIG. 2 illustrates an exploded top perspective view of an embodiment of the condom packaging 10, comprising a container 12 with a flat round bottom 18 and cylindrical side wall 20 with an upper rim 22 for attaching the removable top 14. A condom 24 is situated to fit within the container 12 and is configured such that the condom 24 may be directly placed with the container 12 upon a penis without coming in contact with the end user. The condom 24 is further configured in the container 12 to allow for direct unrolling of the condom 24 onto the penis, such that the condom 24 cannot be mishandled or misfitted. FIG. 2 further depicts the removable top 14 and edge 16, whereas the edge 16 protrudes beyond the upper rim 22 of the container 12 for ease of handling and removal of the removable top 14 by an end user.

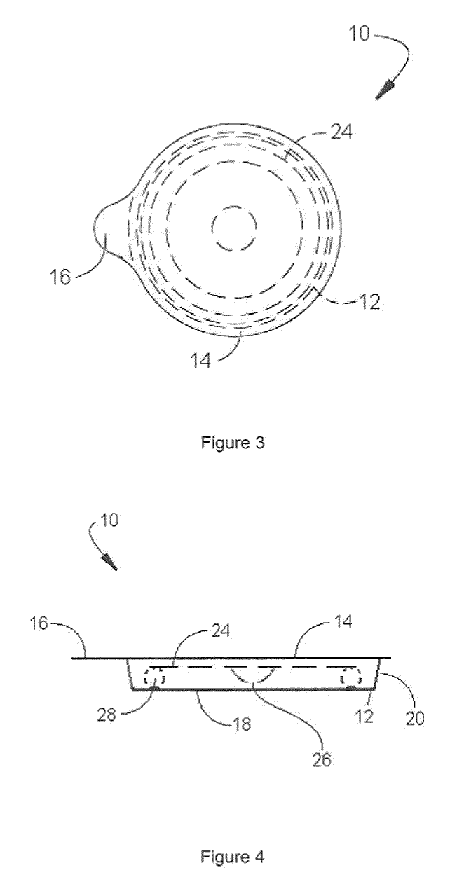

FIG. 3 illustrates a top diaphanous view of an embodiment of the condom packaging 10, comprising a container 12, a removable top 14 attached to the container 12, as well as an edge 16 protruding beyond the upper rim 22 of the removable top 14 for ease of handling and removal of the removable top 14 by an end user.

FIG. 4 illustrates a side sectional view of an embodiment of the condom packaging 10, comprising a condom 24, removable top 14 with edge 16, and a container 12, depicting the flat round bottom 18 and cylindrical side wall 20. The illustration further details the configuration of the condom 24 with the condom head 26 situated towards the round bottom 18 of the container 12, and the condom rolls 28 configured to roll out away from the round bottom 18 of the container 12, such that when applied to a penis, the condom head 26 protrudes from the head of the penis and the condom rolls 28 may only be unrolled towards the base of the penis.

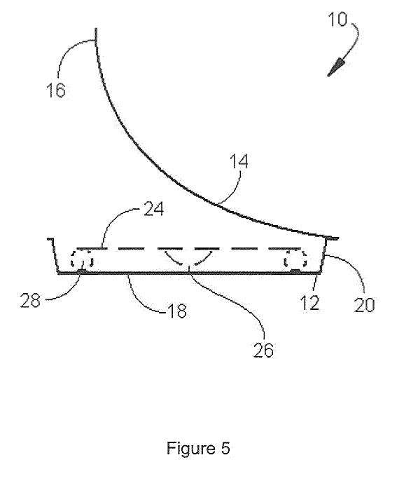

FIG. 5 illustrates a side sectional view of an embodiment of the condom packaging 10, with the removable top 14 being partially removed. Similar to FIG. 4, FIG. 5 also depicts the condom 24, removable top 14 with edge 16, and a container 12 with a flat round bottom 18 and cylindrical side wall 20. The illustration also details the configuration of the condom 24 with the condom head 26 situated towards the round bottom 18 of the container 12, and the condom rolls 28 configured to roll out away from the round bottom 18 of the container 12. FIG. 5 also depicts the partial removal of removable top. 14 initiated by pulling on the edge 16 detaching the removable top 14 from the container 12, thus exposing the condom 24 for application, with the container 12, to the penis.

In operating the condom packaging 10 is container 12 is handled by one hand of the end user, while the other hand pulls up on the removable top 14 exposing the condom 24. The removable top 14 is entirely removed and discarded, and the container 12 containing the condom 24 is placed on the head of the penis. All the while care is taken not to contact the condom with the end user's hands, thus reducing any incidents of tears, rips or other physical damage to the condom 24. The rolled ends of the condom, or condom rolls 28, are unrolled upon and towards the base of the end user's penis until the condom 24 is fitted upon the penis. The container 12 is discarded and the end user is free to commence intercourse.

In various embodiments, the removable top 14 may be luminous to aid the end user in locating and removing the removable top, as well as properly fitting the condom without disorienting the condom.

Various embodiments of the subject matter are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the present subject matter known to the applicant at this time of filing the application has been presented and is intended for the purposes of illustration and description. The present description is not intended to be exhaustive nor limit the subject matter to the precise form disclosed and many modifications and variations are possible in the light of the above teachings. The embodiments described serve to explain the principles of the subject matter and its practical application and to enable others skilled in the art to utilize the present subject matter in various embodiments and with various modifications as are suited to the particular use contemplated. Therefore, it is intended that the present subject matter not be limited to the particular embodiments disclosed for carrying out the present subject matter.

While particular embodiments of the present subject matter have been shown and described, it will be obvious to those skilled in the art that, based upon the teachings herein, changes and modifications may be made without departing from the present subject matter and its broader aspects and, therefore, the appended claims are to encompass within their scope all such changes and modifications as are within the true spirit and scope of the present subject matter. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.)

What is claimed is:

1. A condom package for dispensing a condom, comprising:
a container having a cylindrical shape with a round bottom wall, a cylindrical side wall with an upper rim, and an open top, wherein the diameter of the bottom wall is configured to encompass the periphery of a rolled condom and the height of the side wall is configured to encompass the periphery of the rolled condom; and
a removable top sealably attached to the upper rim of the side wall of the container, with the removable top covering the container.

2. The condom package according to claim 1, wherein the removable top has an edge extending beyond the upper rim of the side wall for ease of removing the removable top.

3. The condom package according to claim 1, wherein the removable top is sealably attached by an adhesive.

4. The condom package according to claim 1, wherein the removable top is selected from the group consisting of a pop-top, a pull-tab, a plastic lid, a peel-off top and combinations thereof.

5. The condom package according to claim 1, wherein the removable top is constructed of a material selected from the group consisting of a plastic, an elastomer, rubber, an elastic polymer, a metal alloy, derivatives thereof and combinations thereof.

6. The condom package according to claim 1, wherein the container is constructed of a material selected from the group consisting of a plastic, an elastomer, rubber, an elastic polymer, a metal alloy, derivatives thereof and combinations thereof.

7. The condom package according to claim 1, wherein the removable top is luminous.

8. In combination, the condom package according to claim 1; and condom sealed in the container, wherein the condom is configured for direct placement upon a penis and rolling onto the penis.

9. A method for applying a condom, comprising:
providing a condom package for dispensing a condom, comprising:
a container having a cylindrical shape with a round bottom wall, a cylindrical side wall with an upper rim, and an open top, wherein the diameter of the bottom wall encompasses the periphery of a rolled condom and the height of the side wall encompasses the periphery of a rolled condom,
a removable top sealably attached to the upper rim of the side wall of the container, with the removable top covering the container, and
a rolled condom housed in the container and configured for direct placement upon a penis and unrolling onto the penis;
removing the removable top from the condom package;
placing the container containing the rolled condom upon the penis;
unrolling the rolled condom onto the penis; and
removing the container from the penis.

10. The method of claim 9, wherein the removable top of the condom package has an edge extending beyond the upper rim of the side wall for ease of removing the removable top.

11. The method of claim 9, wherein the removable top of the condom package is sealably attached by an adhesive.

12. The method of claim 9, wherein the removable top of the condom package is selected from the group consisting of a pop-top, a pull-tab, a plastic lid, a peel-off top and combinations thereof.

13. The method of claim 9, wherein the removable top of the condom package is constructed of a material selected from the group consisting of a plastic, an elastomer, rubber, an elastic polymer, a metal alloy, derivatives thereof and combinations thereof.

14. The method of claim 9, wherein the container of the condom package is constructed of a material selected from the group consisting of a plastic, an elastomer, rubber, an elastic polymer, a metal alloy, derivatives thereof and combinations

15. The method of claim 9, wherein the removable top of the condom package is luminous.
